(19) United States
(12) Patent Application Publication
(10) Pub. No.: US 2018/0028335 A1
(43) Pub. Date: Feb. 1, 2018

(54) MEMORY AND TIME EFFICIENT RESAMPLING FOR 3D PRINTING FROM VOXELS

(71) Applicant: FOVIA, INC., Palo Alto, CA (US)

(72) Inventor: Kevin KREEGER, Palo Alto, CA (US)

(21) Appl. No.: 15/659,444

(22) Filed: Jul. 25, 2017

Related U.S. Application Data
(60) Provisional application No. 62/368,607, filed on Jul. 29, 2016.

(51) Int. Cl. A61F 2/50; G09G 5/36; G06T 3/40; G06T 1/60; G06T 5/00

(52) U.S. Cl. CPC A61F 2/5046; G06T 1/60; G06T 5/006; G06T 3/40; G09G 5/363

(57) ABSTRACT

According to one aspect, systems and processes for resampling data for 3D printing from voxels are provided. In an exemplary process, a native resolution dataset representing an object to be printed is received. A first slice of data from the native resolution dataset is up-sampled, and first up-sampled data is communicated to a 3D printer for printing. Next, a second slice of data from the native resolution dataset is up-sampled, and second up-sampled data is communicated to the 3D printer for printing. Furthermore, the second slice of data may be up-sampled subsequent to the up-sampling of the first slice of data or subsequent to the communicating of the first up-sampled data to the 3D printer.

### CROSS REFERENCE TO RELATED APPLICATIONS

This application claims priority to U.S. Provisional Patent Application No. 62/368,607, filed on Jul. 29, 2016, for which the entire content is hereby incorporated by reference in its entirety for all purposes.

### FIELD

This relates generally to the field of 3D printing, and in particular, to 3D printing of medically-scanned data at a resolution different from the resolution a 3D printer is configured for printing.

### BACKGROUND

Current systems for performing 3D printing typically utilize meshes of polygons as the model for the object to be printed. For example, 3D printing originated from Computer Aided Design applications typically creates polygonal meshes. Therefore, the interface to 3D printers utilizes these meshes as the input model for the object to be printed. However, typical 3D printers themselves lay down material (or fuse material or other “additive” methods) on “planes” across the z-dimension. In particular, the material is laid down either via raster pattern (e.g., move to x₁,y₁, deposit material from there in a line to x₂,y₂, move to x₃,y₃, etc.) or on a 2D grid (e.g., a list of all xᵢ,yᵢ that should have material), where a stack of 2D grids are substantially similar to a 3D volumetric grid of data.

When 3D printing medically-scanned data, such as computer tomography (CT) or magnetic resonance imaging (MRI) data, as well as other 3D regular grids and 3D data outside of the medical field, the model is in the 3D volumetric grid format already. However, because of the interfaces to the 3D printers, the model of the object to be printed is first converted to a polygonal mesh (e.g., using marching cubes algorithm or other algorithms), which is then “sliced” back into layers (of either raster or pixel data).

One approach to adapt medically-scanned data to 3D printing includes a method of printing volumetric data using slices of pixels directly from a volumetric mode, wherein neither conversion from model to polygonal mesh, nor from polygonal mesh into sliced layers, is required. One challenge with this method is that medically-scanned data is typically at a much lower resolution than a printer is designed for printing. For example, typical medical CT data is scanned at between about 500 microns and about 1000 microns (0.5 mm to 1.0 mm) in resolution, while the printers print at about 20-micron resolution. Sending slices of data from medical CT scans may thus generate very small printed objects if one voxel of the medical data represents one pixel of one slice on the printer. In the case of medical CT data, objects may be printed at, for example, at least 25 times smaller than true size. If the desire is to print the objects larger (e.g., approximately the true size of an object or within an order of magnitude of true size) such that it is easier for a user to see, it may require converting the medical scanned models into much higher resolution datasets. However, existing techniques for creating and sending high resolution pixel slices to 3D printers are inefficient. Thus, improved techniques for adapting medically-scanned data to 3D printing, such as optimization in time and memory, are desired.

### SUMMARY

According to one aspect of the present disclosure, a method for resampling data for 3D printing from voxels is provided. In some embodiments, the method includes, at an electronic device having a processor and memory, receiving a native resolution dataset representing an object to be printed. The method can further include up-sampling a first slice of data from the native resolution dataset. The method can further include communicating the first up-sampled data to a 3D printer for printing. The method can further include up-sampling a second slice of data from the native resolution dataset. The method can further include communicating the second up-sampled data to the 3D printer for printing. Furthermore, the second slice of data may be up-sampled subsequent to the up-sampling of the first slice of data or subsequent to the communicating of the first up-sampled data to the 3D printer.

In some embodiments, the up-sampling of the first slice of data includes interpolating data and classifying data, wherein the interpolating is performed prior to the classifying. In some embodiments, classifying data includes mapping a scalar field of the native resolution dataset to one or more printable material properties. In some embodiments, the one or more printable material properties include at least one of color, transparency, and stiffness. In some embodiments, the up-sampling of the first slice of data or the second slice of data is based on at least one of a nearest neighbor technique, a linear interpolation technique, a cubic interpolation technique, and a quadratic interpolation technique. In some embodiments, the native resolution dataset includes at least one of computer tomography data or magnetic resonance imaging data.

In some embodiments, a system for resampling data for 3D printing from voxels is provided. In some embodiments, the system includes a display, one or more processors, and a memory storing one or more programs, wherein the one or more programs include instructions configured to be executed by the one or more processors, causing the one or more processors to perform operations. The operations may include receiving a native resolution dataset representing an object to be printed. The operations may further include up-sampling a first slice of data from the native resolution dataset. The operations may further include communicating the first up-sampled data to a 3D printer for printing. The operations may further include up-sampling a second slice of data from the native resolution dataset. Furthermore, the operations may include communicating the second up-sampled data to the 3D printer for printing. The second slice of data may further be up-sampled subsequent to the up-sampling of the first slice of data or subsequent to the communicating of the first up-sampled data to the 3D printer.

In some embodiments, the up-sampling of the first slice of data comprises interpolating data and classifying data, where the interpolating is performed prior to the classifying. In some embodiments, classifying data comprises: mapping a scalar field of the native resolution dataset to one or more printable material properties. In some embodiments, the one or more printable material properties include at least one of color, transparency, and stiffness. In some embodiments, the up-sampling of the first slice of data or the second slice of data is based on at least one of a nearest neighbor technique, a linear interpolation technique, a cubic interpolation technique, and a quadratic interpolation technique. In some embodiments, the native resolution dataset includes at least one of computer tomography data or magnetic resonance imaging data.

In some embodiments, an additional method for resampling data for 3D printing from voxels is provided. The method may include, at an electronic device having a processor and memory, subsampling a native resolution dataset based on an octree data structure, wherein the native resolution dataset is associated with an object to be printed; and communicating an output slice of the subsampled data to a 3D printer. The method may include generating sub-voxel layers to the octree associated with an output slice. Furthermore, the sub-voxel layers may be generated at a predetermined size of resolution of the output slice. In some embodiments, subsampling a native resolution dataset may include generating a second octree data structure based on the native resolution dataset, storing the second octree data structure, and deleting the native resolution dataset after generating and storing the second octree data structure. Furthermore, the native resolution dataset may be subsampled based on at least one of a nearest neighbor technique, a linear interpolation technique, a cubic interpolation technique, and a quadratic interpolation technique. In some embodiments, the native resolution dataset includes at least one of computer tomography data or magnetic resonance imaging data. In some embodiments, the method includes generating a copy of the native resolution dataset based on the octree data structure. In some embodiments, the method includes determining an isosurface location of binary data within the native resolution dataset.

### BRIEF DESCRIPTION OF THE DRAWINGS

FIG. 1 illustrates an exemplary system for resampling for 3D printing from voxels.

FIG. 2 illustrates an exemplary process for resampling for 3D printing from voxels.

FIG. 3 illustrates an exemplary process for utilizing a speedup procedure in resampling for 3D printing from voxels.

### DETAILED DESCRIPTION

In general, techniques described in this disclosure provide for memory and time efficient resampling for 3D printing from voxels. Furthermore, the present invention may take the form of an entirely software embodiment, entirely hardware embodiment, or a combination of software and hardware embodiments. Even further, the present invention may take the form of a computer program product contained on a computer-readable storage medium, where computer-readable code is embodied on the storage medium. The computer-readable storage medium can be non-volatile. In another embodiment, the present invention may take the form of computer software implemented as a service (SaaS). Any appropriate storage medium may be utilized, such as optical storage, magnetic storage, hard disks, or CD-ROMs.

In the following description of the techniques and examples, reference is made to the accompanying drawings in which it is shown by way of illustration specific examples that can be practiced. It is to be understood that other examples can be practiced and structural changes can be made without departing from the scope of the disclosure.

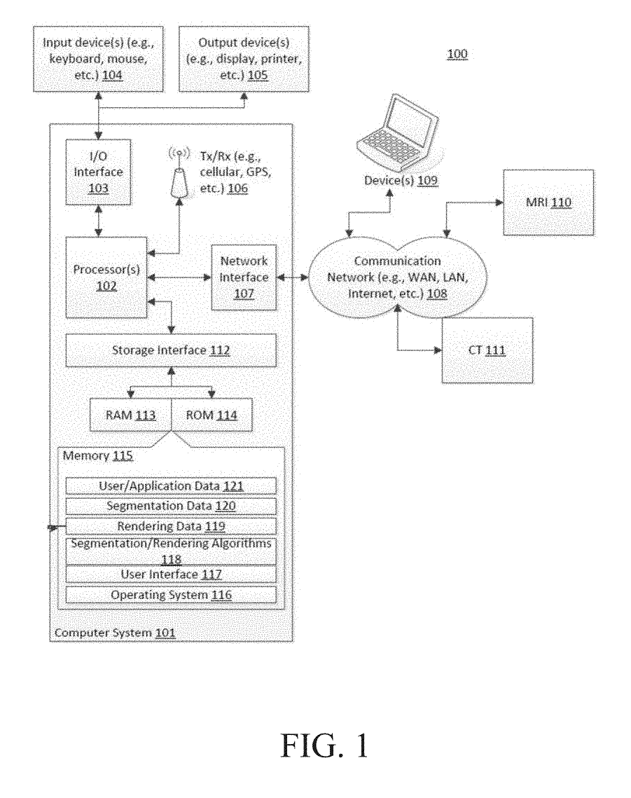

FIG. 1 illustrates an exemplary system 100 for resampling for 3D printing from voxels, consistent with some embodiments of the present disclosure. In some embodiments, system 100 may include a computer system 101, input devices 104, output devices 105, devices 109, MRI system 110, and CT system 111. It is appreciated that one or more components of system 100 can be separate systems or can be integrated systems. In some embodiments, computer system 101 may comprise one or more central processing units (“CPU” or “processor(s)”) 102. Processor(s) 102 may comprise at least one data processor for executing program components for executing user- or system-generated requests. A user may include a person, a person using a device such as those included in this disclosure, or such a device itself. The processor may include specialized processing units such as integrated system (bus) controllers, memory management control units, floating point units, graphics processing units, digital signal processing units, etc. The processor may include a microprocessor, such as AMD Athlon, Duron or Opteron, ARM's application, embedded or secure processors, IBM PowerPC, Intel's Core, Itanium, Xeon, Celeron or other line of processors, etc. The processor 102 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor(s) 102 may be disposed in communication with one or more input/output (I/O) devices via I/O interface 103. I/O interface 103 may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.11 a/b/g/n/x, Bluetooth, cellular (e.g., code-division multiple access (CDMA), high-speed packet access (HSPA+), global system for mobile communications (GSM), long-term evolution (LTE), WiMax, or the like), etc.

Using I/O interface 103, computer system 101 may communicate with one or more I/O devices. For example, input device 104 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, electrical pointing devices, etc. Output device 105 may be a printer, fax machine, video display (e.g., cathode ray tube (CRT), liquid crystal display (LCD), light-emitting diode (LED), plasma, or the like), audio speaker, etc. In some embodiments, a transceiver 106 may be disposed in connection with the processor(s) 102. The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc.

In some embodiments, processor(s) 102 may be disposed in communication with a communication network 108 via a network interface 107. Network interface 107 may communicate with communication network 108. Network interface 107 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. Communication network 108 may include, without limitation, a direct interconnection, local area network (LAN), wide area network (WAN), wireless network (e.g., using Wireless Application Protocol), the Internet, etc. Using network interface 107 and communication network 108, computer system 101 may communicate with devices 109. These devices may include, without limitation, personal computer(s), server(s), fax machines, printers, scanners, various mobile devices such as cellular telephones, smartphones (e.g., Apple iPhone, Blackberry, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, computer system 101 may itself embody one or more of these devices.

In some embodiments, using network interface 107 and communication network 108, computer system 101 may communicate with MRI system 110, CT system 111, or any other medical imaging systems. Computer system 101 may communicate with these imaging systems to obtain images for segmentation. Computer system 101 may also be integrated with these imaging systems.

In some embodiments, processor 102 may be disposed in communication with one or more memory devices (e.g., RAM 113, ROM 114, etc.) via a storage interface 112. The storage interface may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, flash devices, solid-state drives, etc.

The memory devices may store a collection of program or database components, including, without limitation, an operating system 116, user interface application 117, segmentation and rendering algorithm 118, rendering data 119, segmentation data 120, user/application data 121 (e.g., any data variables or data records discussed in this disclosure), etc. Operating system 116 may facilitate resource management and operation of computer system 101. Examples of operating systems include, without limitation, Apple Macintosh OS X, Unix, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. User interface 117 may facilitate display, execution, interaction, manipulation, or operation of program components through textual or graphical facilities. For example, user interfaces may provide computer interaction interface elements on a display system operatively connected to computer system 101, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, Javascript, AJAX, HTML, Adobe Flash, etc.), or the like.

In some embodiments, computer system 101 may implement segmentation and rendering algorithms 118. Segmentation and rendering algorithms 118 include, for example, a minimum/maximum connected component region grow segmentation algorithm, a minimum/maximum connected component with gradient weighted stopping influence algorithm, a level-set algorithm, a watershed algorithm, a gradient-based algorithm, a floodfill algorithm, a thresholding algorithm, a blow/suck/lasso/balloon-physics-based algorithm, an Otsu's method of histogram clustering algorithm, a Vesselness algorithm, an one-click-bone-removal algorithm, a statistical algorithm, an example-patch Al algorithm, or an algorithm with or without dilation and erode. One or more of these algorithms may be used for segmenting images. Computer system 101 may also store the segmentation data 120 and rendering data 119. For example, memory 115 and/or a frame buffer (not shown) may be used for storing rendering data 119.

In some embodiments, computer system 101 may store user/application data 121, such as data, variables, and parameters (such as segmentation parameters) as described in this disclosure. Such databases may be implemented as fault-tolerant, relational, scalable, secure databases such as Oracle or Sybase. Alternatively, such databases may be implemented using standardized data structures, such as an array, hash, linked list, struct, structured text file (e.g., XML), table, or as object-oriented databases (e.g., using ObjectStore, Poet, Zope, etc.). Such databases may be consolidated or distributed, sometimes among the various computer systems discussed above in this disclosure. It is to be understood that the structure and operation of any computer or database component may be combined, consolidated, or distributed in any working combination.

Disclosed embodiments describe systems and methods for resampling for 3D printing from voxels. The illustrated components and steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items.

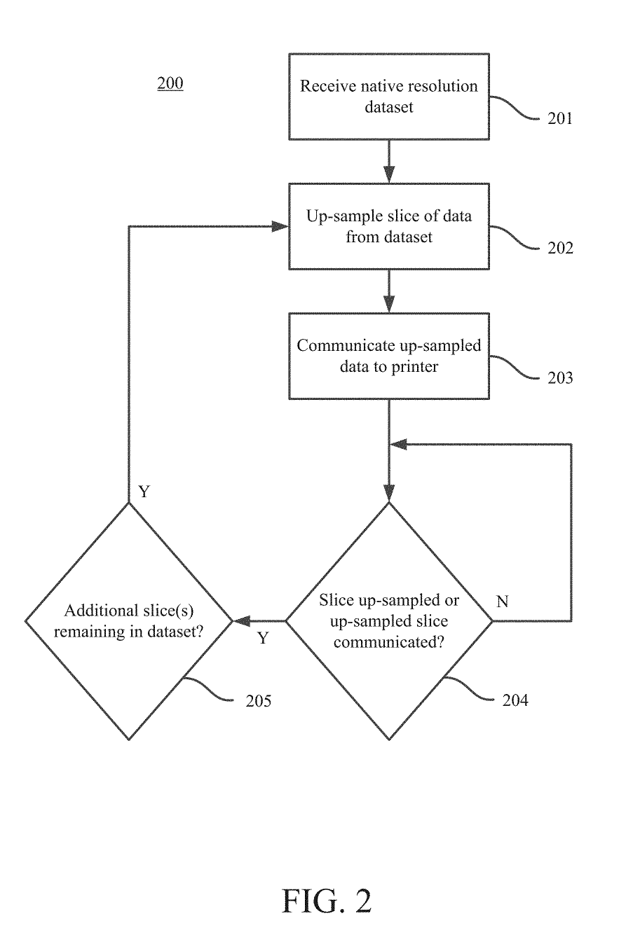

FIG. 2 illustrates an exemplary process 200 for up-sampling a native resolution dataset. In some embodiments, at a first step 201, a native resolution dataset is received. In one example, a dataset may include a 512×512×2000 slice medical dataset, which may include, for example, 62 MB of data (assuming 1 bit per voxel). A conventional method is to make a copy of the dataset at a higher resolution via subsampling. There are a number of sampling techniques to accomplish this, including nearest neighbor, which creates blocky looking objects. When using a nearest neighbor technique, each pixel is replaced with a plurality of pixels having the same attributes. Using such a technique, the image output is larger, but may exhibit undesirable characteristics, such as rough edges or boundary lines. When using a linear interpolation technique, known data points, such as attributes of a pixel, are used to approximate values between the known data points. Linear interpolation may generate smoother but “faceted” models where faces are planes. When using a higher order interpolation technique, such as cubic or quadratic interpolation techniques, smoother, non-planer surfaces of objects are created. Using a higher order interpolation technique, intricate shapes such ovals, waves, or other curved surfaces may be approximated. All of these methods, however, may require much more storage, effectively rendering them impractical, undesirable, and/or unusable. For example, assuming a typical medical dataset resolution of 0.8 mm×0.8 mm×0.8 mm and printing at approximately 20-micron resolution, an “up-sampled to 20-micron dataset” would consume 64,000 times more memory than the dataset, or about 4 TB .

In some embodiments, an improved system as described herein can include only up-sampling the native resolution dataset (e.g., CT data at a 500-1000 micron resolution) to the printer-resolution (e.g., printer data at a 20 micron resolution) immediately before sending it to the printer. Because printers operate by using slices and slices are sent one at a time, in one example the system only up-samples the data for the next slice before sending to the printer. Sampling in this manner may require additional memory to be no more than, for example, 50 MB over the original dataset, in the above example. As a result, the slices can be generated in a time-efficient manner, because the slices are created in substantially real time (e.g., “on the fly”).

With reference back to FIG. 2, in some embodiments, at step 202, a slice of data from the dataset can be up-sampled. In general, up-sampling may refer to the process of increasing the sampling rate of a target, such as a signal or other data. For example, up-sampling may include introducing additional information into a dataset in order to approximate a hypothetical dataset. The hypothetical dataset may, for example, represent a dataset that would have been obtained if the original dataset had been sampled at a higher sampling rate. In some embodiments, at step 203, the up-sampled data can be communicated to a printer. For example, the data may be communicated via a wired or wireless connection. In some embodiments, at step 204, a determination can be made as to whether the slice was up-sampled and/or whether the up-sampled slice was communicated to the printer. For example, a determination as to whether the slice was up-sampled may include accessing system memory to determine whether up-sampled data corresponding to the up-sampled slice has been processed and maintained in system memory. As another example, a determination as to whether the up-sampled slice was communicated to the printer may include accessing printer memory to determine whether up-sampled data corresponding to the up-sampled slice has been received and maintained in printer memory.

Referring back to FIG. 2, in accordance with a determination that either the slice was up-sampled or the up-sampled slice was communicated to the printer, the process 200 advances to step 205. At step 205, a determination can be made as to whether the dataset includes one or more additional slices to be up-sampled. For example, such determination may include comparing slices in the dataset to up-sampled data to determine slices of data in the dataset that are not associated with corresponding up-sampled data. In some embodiments, in accordance with a determination that one or more additional slices of data are remaining in the dataset at step 205, the process 200 can repeat step 202 to up-sample the additional slice. When up-sampling, in some embodiments, it is generally desired that dataset is first interpolated and then classified. Interpolation includes, for example, up-sampling the native resolution dataset. Classification includes, for example, mapping the scalar field of the input CT or MR data to printable material properties (color, transparency, stiffness, etc.). The process of first interpolating and then classifying thus provides a smooth result across the resulting property, instead of a blocky one at the native resolution.

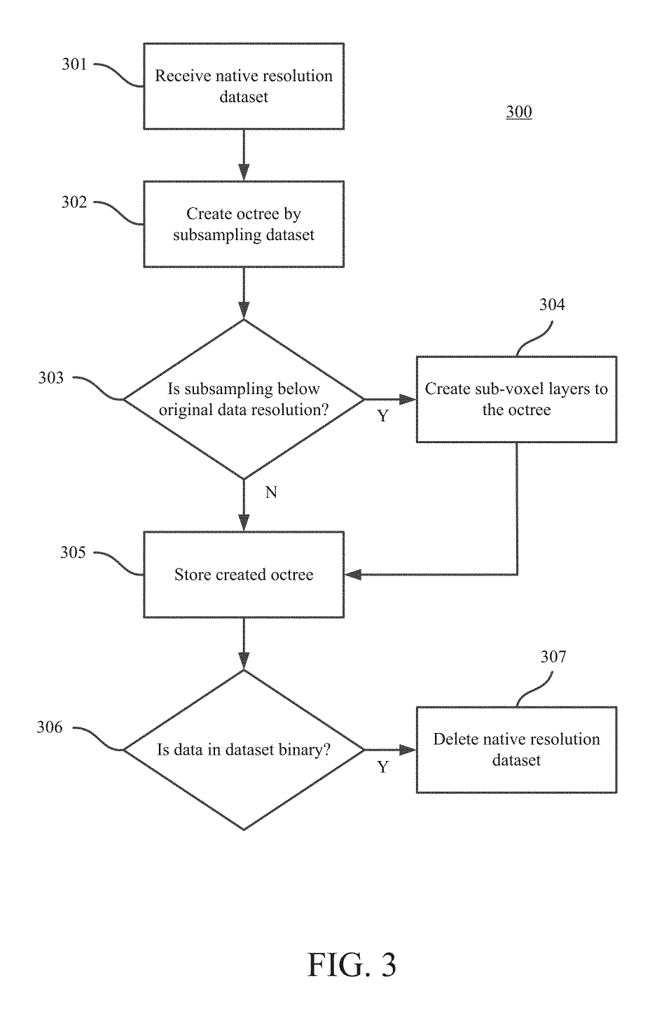

FIG. 3 illustrates an exemplary process 300 for generating data based on an efficient data-structure. An efficient data-structure can increase the speed or efficiency of storing and retrieving information, for example. In some embodiments, process 300 may use an octree data structure to increase the speed or efficiency of subsampling the dataset at high resolution slice locations. An octree data structure (or an “octree”) may allow efficient access to data, especially when the data contains a large amount of contiguous similar regions (such as in binary 3D models-to-print). With reference to FIG. 3, in some embodiments, a native resolution dataset can be received at step 301. In some embodiments, an octree is generated by subsampling the received native resolution dataset at step 302. In general, subsampling can refer to the process of reducing the sampling rate of a target, such as a signal or other set of information. In some embodiments, when subsampling below the original data resolution, one or more sub-voxel layers to the octree may be generated as an additional speedup to the octree. In some embodiments, at step 303, a determination is made as to whether a resolution of the subsampling is below the original data resolution. For example, a subsampling resolution may be set at a 20 micron resolution, whereas the resolution of the native resolution dataset may be a 500 micron resolution. In this example, a positive determination would result, in order to signify that a resolution of the subsampling is below the original data resolution.

In some embodiments, in accordance with a determination at step 303 that subsampling resolution is below the original dataset resolution, sub-voxel layers to the octree are generated at step 304. For example, sub-voxel layers may be generated down to the size of the desired resolution of the output slices. Sub-voxel layers may be generated, for example, using a nearest neighbor approach, a linear interpolation approach, a cubic interpolation approach, or a quadratic interpolation approach. This process may result in faster, more efficient slice creation at a very small increase in memory size, since only the octree needs to be created for the sub-voxel layers. For example, utilizing the octree to create sub-voxel layers may require minimal memory usage over the original dataset (e.g., 50 MB), similar to the above example.

In some embodiments, upon completion of the generation of sub-voxel layers to the octree at step 304, process 300 may advance to step 305, where the generated octree is stored. Alternatively, in some embodiments, in accordance with a determination at step 303 that subsampling resolution is greater than or equal to the resolution of the native resolution dataset, the octree is stored at step 305 without creating sub-voxel layers at step 304. In some embodiments, a determination is made at step 306 as to whether the data is binary. For example, data within the native resolution dataset may be parsed and analyzed using a heuristic for determining binary file types. In accordance with a determination at step 306 that the data in the dataset is binary, the received native resolution dataset may be deleted at step 307, such that the octree is retained without the native resolution dataset. Such deletion is advantageous in that the original data can be re-created by the octree data structure.

In the above-described exemplary process 300, interpolating of binary data can be smoothed via various techniques. For example, data can be converted to higher bit-resolution (e.g., from 1000 micron resolution to 20 micron resolution). In one example, a value for “on” may be set to “255” stored in one byte and a value for “off” may be set to “0.” Interpolation can occur between the “on” and “off” values prior to smoothing the interpolation. Furthermore, for example, a determination may be made where an isosurface is located (e.g., at “128”) and the determined isosurface location may be used as the binary boundary of the object. This process may be performed at interpolation time. As a result, extra memory may not be required to store the data.

Various embodiments described herein can be used for various applications. For example, the embodiments may be utilized in 3D printing, such as when different material properties are modeled across traditional Polygonal Mesh computer-aided design models. For example, such properties may be the same or similar to those described above, but rather than mapping properties from CT or MR data, the designer may create the properties to the model. Thus, based on the foregoing disclosure, improvements are provided over conventional systems. Specifically, conventional systems compute, store, and process volumetric data at a very high resolution, resulting in increased resource usage. The improvements discussed herein allow for a user to choose an appropriate resolution to achieve the desired effect of material changing, and further allow for storage of the voxel data at that resolution. Subsequently, this method can be used to resample to the printer resolution before sending data to the printer.

The description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the present technology. Thus, the disclosed technology is not intended to be limited to the examples described herein and shown, but is to be accorded the scope consistent with the claims.

What is claimed is:

1. A method for resampling data for 3D printing from voxels, the method comprising:
at an electronic device having a processor and memory:
receiving a native resolution dataset representing an object to be printed;
up-sampling a first slice of data from the native resolution dataset;
communicating the first up-sampled data to a 3D printer for printing;
up-sampling a second slice of data from the native resolution dataset; and
communicating the second up-sampled data to the 3D printer for printing, wherein the second slice of data is up-sampled subsequent to the up-sampling of the first slice of data or subsequent to the communicating of the first up-sampled data to the 3D printer.

2. The method of claim 1, wherein the up-sampling of the first slice of data comprises interpolating data and classifying data, wherein the interpolating is performed prior to the classifying.

3. The method of claim 2, wherein classifying data comprises:
mapping a scalar field of the native resolution dataset to one or more printable material properties.

4. The method of claim 3, wherein the one or more printable material properties include at least one of color, transparency, and stiffness.

5. The method of claim 1, wherein the up-sampling of the first slice of data or the second slice of data is based on at least one of a nearest neighbor technique, a linear interpolation technique, a cubic interpolation technique, and a quadratic interpolation technique.

6. The method of claim 1, wherein the native resolution dataset includes at least one of computer tomography data or magnetic resonance imaging data.

7. A system for resampling data for 3D printing from voxels, the system comprising:
a display;
one or more processors; and
a memory storing one or more programs, wherein the one or more programs include instructions configured to be executed by the one or more processors, causing the one or more processors to perform operations comprising:
receiving a native resolution dataset representing an object to be printed;
up-sampling a first slice of data from the native resolution dataset;
communicating the first up-sampled data to a 3D printer for printing;
up-sampling a second slice of data from the native resolution dataset; and
communicating the second up-sampled data to the 3D printer for printing, wherein the second slice of data is up-sampled subsequent to the up-sampling of the first slice of data or subsequent to the communicating of the first up-sampled data to the 3D printer.

8. The system of claim 7, wherein the up-sampling of the first slice of data comprises interpolating data and classifying data, wherein the interpolating is performed prior to the classifying.

9. The system of claim 8, wherein classifying data comprises:
mapping a scalar field of the native resolution dataset to one or more printable material properties.

10. The system of claim 9, wherein the one or more printable material properties include at least one of color, transparency, and stiffness.

11. The system of claim 7, wherein the up-sampling of the first slice of data or the second slice of data is based on at least one of a nearest neighbor technique, a linear interpolation technique, a cubic interpolation technique, and a quadratic interpolation technique.

12. The system of claim 7, wherein the native resolution dataset includes at least one of computer tomography data or magnetic resonance imaging data.

13. A method for resampling data for 3D printing from voxels, the method comprising:
at an electronic device having a processor and memory:
subsampling a native resolution dataset based on an octree data structure, wherein the native resolution dataset is associated with an object to be printed; and
communicating an output slice of the subsampled data to a 3D printer.

14. The method of claim 13, further comprising:
generating sub-voxel layers to the octree associated with an output slice.

15. The method of claim 14, wherein the sub-voxel layers are generated at a predetermined size of resolution of the output slice.

16. The method of claim 13, wherein subsampling a native resolution dataset further comprises:
generating a second octree data structure based on the native resolution dataset;
storing the second octree data structure; and
deleting the native resolution dataset after generating and storing the second octree data structure.

17. The method of claim 13, wherein the native resolution dataset is subsampled based on at least one of a nearest neighbor technique, a linear interpolation technique, a cubic interpolation technique, and a quadratic interpolation technique.

18. The method of claim 13, wherein the native resolution dataset includes at least one of computer tomography data or magnetic resonance imaging data.

19. The method of claim 13, further comprising:
generating a copy of the native resolution dataset based on the octree data structure.

20. The method of claim 13, further comprising:
determining an isosurface location of binary data within the native resolution dataset.
